(19) United States
(12) Patent Application Publication
(10) Pub. No.: US 2010/0331631 A1
(43) Pub. Date: Dec. 30, 2010

(54) OXYGEN SATURATION EAR SENSOR DESIGN THAT OPTIMIZES BOTH ATTACHMENT METHOD AND SIGNAL QUALITY

(75) Inventor: Scott MacLaughlin, Boulder, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/495,545

(22) Filed: Jun. 30, 2009

(51) Int. Cl. A61B 5/1455; A61B 5/00

(52) U.S. Cl. 600/301; 600/344; 600/324

(57) ABSTRACT

A system is provided that includes an ear sensor and an external device. The ear sensor includes a sensing component with sensors for sensing various physiological parameters. The ear sensor also includes a retaining component configured to retain the ear sensor to the ear of a wearer. As the retaining component retains the ear sensor to the ear, the sensing component may be configured to have an optimal surface contact between the sensors and the ear tissue, such that an improved physiological signal may be obtained. In some embodiments, the improved physiological signal may result in physiological data, which may be displayed and organized in the external device.

### BACKGROUND

The present disclosure relates generally to medical devices and, more particularly, to sensors used for sensing physiological parameters of a patient.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light to a patient's tissue and that photoelectrically detects the absorption, scattering, and/or reflection of the transmitted light in such tissue. The absorption, scattering, and/or reflection of the transmitted light that is sensed may also be referred to as a pulse oximetry signal, and sensors may include reflective and/or transmittance-style sensors, depending on whether the pulse oximeter is configured to detect absorbed, scattered, and/or reflected light. One or more of the above physiological characteristics may then be calculated based upon the pulse oximetry signal. More specifically, the light transmitted to the tissue is typically selected to be of one or more wavelengths that may be absorbed, scattered, and/or reflected by the blood in an amount correlative to the amount of the blood constituent present in the blood. The detected pulse oximetry signal may then be used to estimate the amount of blood constituent in the tissue using various algorithms.

One example of a pulse oximetry monitoring device may be an ear sensor that may typically be secured to the ear of the patient to measure a pulse oximetry signal from the ear tissue. The securing device of the ear sensor typically uses the same force to attach the sensor to the patient's ear, as well as to provide surface contact between the sensor and the ear tissue to obtain a pulse oximetry signal. For example, one type of securing device may be a clip that provides the surface contact between the ear lobe and the sensor, and the clip may be further configured to apply sufficient force to retain the sensor to the ear. However, in serving these two functions, the force applied by the securing device may not be optimal in receiving an accurate pulse oximetry signal. For example, the force used to retain the ear sensor to the ear may be greater than the force desired to provide an optimal surface contact between the ear lobe and the sensor. While applying a greater force may serve to retain the device to the ear, the greater force may decrease the quality of the received pulse oximetry signal.

### BRIEF DESCRIPTION OF THE DRAWINGS

Advantages of the disclosure may become apparent upon reading the following detailed description and upon reference to the drawings in which:

FIG. 1 illustrates a perspective view of a pulse oximeter in accordance with an embodiment;

FIG. 1A illustrates an ear sensor wirelessly coupled to a monitor of the pulse oximeter in FIG. 1, in accordance with an embodiment;

FIG. 2 illustrates a simplified block diagram of a pulse oximeter in FIG. 1, according to an embodiment;

FIG. 3 illustrates an ear sensor with a sensing component extending from a processing component of the ear sensor, according to an embodiment;

FIG. 3A illustrates the ear sensor of FIG. 3 worn on an ear, according to an embodiment;

FIG. 3B illustrates a cross sectional view of the sensing component of the car sensor of FIG. 3, coupled to the car lobe, according to an embodiment;

FIG. 4A illustrates an car sensor with a sensing component extending from a retaining component of the ear sensor, according to an embodiment;

FIG. 4B illustrates a different view of the sensing component of the ear sensor of FIG. 4A, according to an embodiment;

FIG. 5 illustrates an ear sensor with a sensing component configured to apply sensors to different areas of the ear and extending from a retaining component of the ear sensor, according to an embodiment;

FIG. 6 illustrates an ear sensor having reflective sensors coupled to the retaining component of the ear sensor, according to an embodiment;

FIG. 7 illustrates an ear sensor having an ear canal member, according to an embodiment;

FIG. 7A illustrates a cross sectional view of an ear sensor of FIG. 7, according to an embodiment;

FIG. 8 illustrates an ear sensor having a control input configured to engage an aural signal corresponding to a pulse oximetry signal, according to an embodiment;

FIG. 9 illustrates a plurality of screens that may be displayed on an external device, illustrating how physiological data from an ear sensor, as illustrated in FIGS. 1-8, for example, may be displayed and organized on the external device, according to an embodiment;

FIG. 10 illustrates a plurality of screens that may be displayed on an external device, illustrating how an application on the external device may display and alert based on physiological data received from an ear sensor, as illustrated in FIGS. 1-8, for example, according to an embodiment;

FIG. 11 illustrates a plurality of screens that may be displayed on an external device, illustrating how an application on the external device may store and organize physiological data received from an ear sensor, as illustrated in FIGS. 1-8, for example, according to an embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to systems and devices that measure physiologic parameters corresponding to blood flow in a patient by emitting light into a patient's tissue with light emitters (e.g., light emitting diodes) and detecting the light (e.g., using a photodetector) after it has passed through or is reflected from the patient's tissue. More specifically, present embodiments are directed to ear sensors configured to receive an improved signal (for use in pulse oximetry, for example) by optimizing a contact between the ear tissue and the light emitters and/or detectors. The ear sensor may also include a retaining component that retains the ear sensor to the patient. While a typical ear sensor may rely on a single force or a single component to perform both the function of providing a surface contact between the sensor and the tissue, as well as the function of retaining the sensor to the ear, an ear sensor of the present techniques may be configured to provide an optimized surface contact between the sensor and the tissue with one force, and retain the ear sensor to the ear with the same or different force. Thus, an improved signal may be received by such an ear sensor. Furthermore, in some embodiments, optimizing the surface contact between the sensor and the tissue may also result in an ear sensor that is more comfortable for the patient.

One or more embodiments of the present techniques are also directed towards displaying and organizing physiological data obtained from an improved pulse oximetry signal. The pulse oximetry signal may be processed to determine the blood-oxygen saturation of hemoglobin in arterial blood (“oxygen saturation” or “SpO2”) and/or the rate of blood pulsations corresponding to each heartbeat of a patient (“pulse rate”). In some embodiments, the ear sensor may also include sensors to obtain other physiological data, such as temperature or blood pressure. The physiological data may be transmitted to be displayed by an external device, in accordance with the present techniques. As will be explained, the external device may display the physiological data, and may allow the user to organize and/or analyze the data.

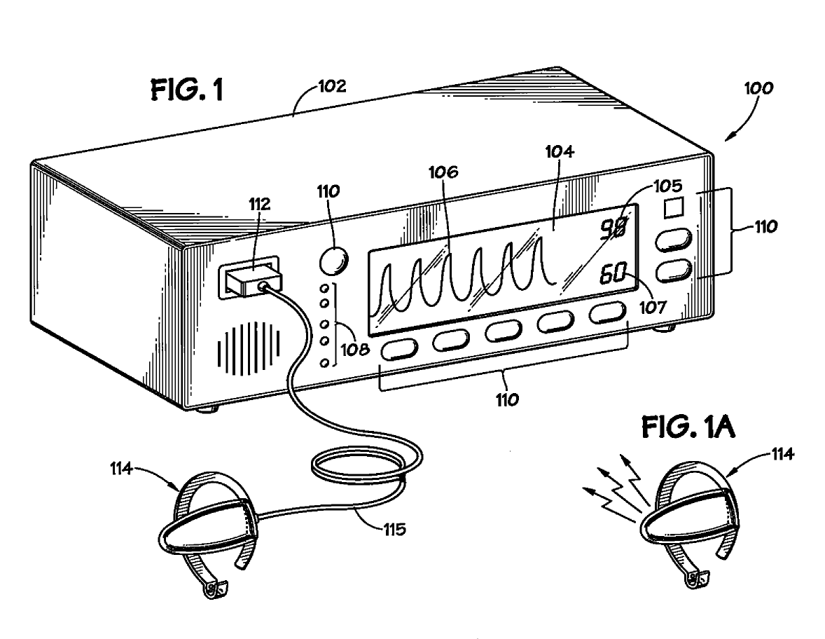

Turning to FIG. 1, a perspective view of a medical device is illustrated in accordance with an embodiment. The medical device may be a pulse oximeter 100. The pulse oximeter 100 may include a monitor 102, such as those available from Nellcor Puritan Bennett LLC. The monitor 102 may be configured to display calculated parameters on a display 104. As illustrated in FIG. 1, the display 104 may be integrated into the monitor 102. However, the monitor 102 may be configured to provide data via a port to a display (not shown) that is not integrated with the monitor 102. The display 104 may be configured to display computed physiological data including, for example, an oxygen saturation percentage 105, a pulse rate 107, and/or a plethysmographic waveform 106. As is known in the art, the oxygen saturation percentage may be a functional arterial hemoglobin oxygen saturation measurement in units of percentage SpO₂, while the pulse rate may indicate a patient's pulse rate in beats per minute. In some embodiments, the monitor 102 may also display other physiological data, such as temperature or blood pressure. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option.

The monitor 102 may further include a sensor port 112. The sensor port 112 may allow for connection to a sensor 114, via a cable 115 which connects to the sensor port 112. The sensor 114 may be of a disposable or a non-disposable type. Furthermore, the sensor 114 may be configured to obtain signals from a patient's ear, and may be referred to as an ear sensor 114, which can be used by the monitor 102 to determine certain physiological characteristics such as the blood-oxygen saturation of hemoglobin in arterial blood, the volume of individual blood pulsations supplying the tissue, and/or the rate of blood pulsations corresponding to each heartbeat of a patient.

In one or more embodiments, the monitor 102 may be a portable device coupled to the ear sensor 114, and information sent from the ear sensor 114 to the portable monitor 102 may be processed and/or calculated to display physiological data on a display 104 integrated into the portable monitor 102. As depicted in FIG. 1A, the ear sensor 114 may communicate with the monitor 102 wirelessly (i.e., without the cable 115), and signals (e.g., pulse oximetry signals) and/or data may be transmitted wirelessly to the monitor 102. Furthermore, one or more functions of the monitor 102 may also be implemented directly in the ear sensor 114. For example, in some embodiments, the ear sensor 114 may include one or more processing components capable of calculating the physiological characteristics from the signals obtained from the patient. In accordance with the present techniques, the ear sensor 114 may be configured to provide optimal contact between a patient, a detector, and/or an emitter, may have varying levels of processing power, and may output data in various stages to a monitor 102 either wirelessly or via the cable 115. For example, in some embodiments, the data output to the monitor 102 may be analog signals, such as detected light signals (e.g., pulse oximetry signals), or processed data. As will be discussed, in some embodiments, the ear sensor 114 may also comprise components, in addition to the sensor components with the emitter 116 and detector 118, configured to retain the body of the ear sensor 114 to ear of the patient 117.

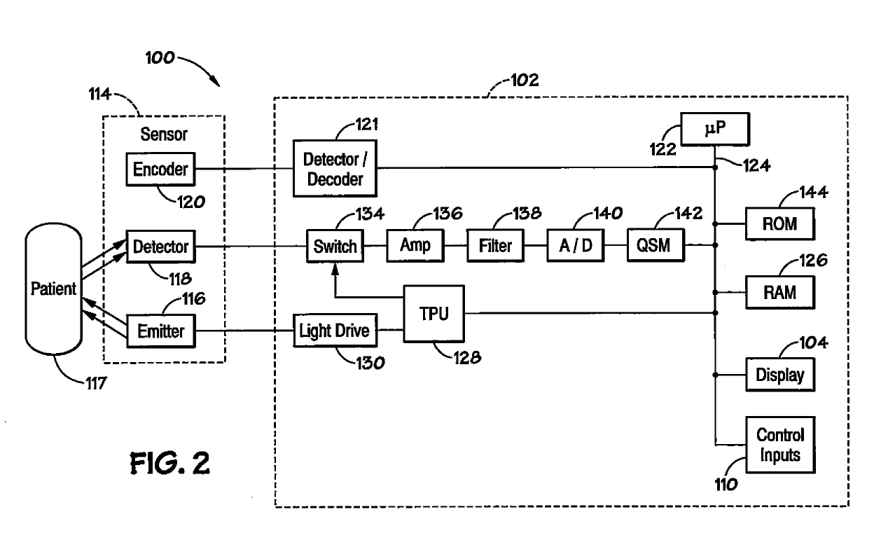

Turning to FIG. 2, a simplified block diagram of a pulse oximeter 100 is illustrated in accordance with an embodiment. Specifically, certain components of the ear sensor 114 and the monitor 102 are illustrated in FIG. 2. The ear sensor 114 may include an emitter 116, a detector 118, and an encoder 120. It should be noted that the emitter 116 may be capable of emitting at least two wavelengths of light, e.g., RED and infrared (IR) light, into the tissue of a patient 117, where the RED wavelength may be between about 600 nanometers (nm) and about 700 nm, and the IR wavelength may be between about 800 nm and about 1000 nm. The emitter 116 may include a single emitting device, for example, with two light emitting diodes (LEDs) or the emitter 116 may include a plurality of emitting devices with, for example, multiple LED's at various locations. Regardless of the number of emitting devices, light from the emitter 116 may be used to measure, for example, water fractions, hematocrit, or other physiologic parameters of the patient 117. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 118 may be an array of detector elements that may be capable of detecting light at various intensities and wavelengths. In one embodiment, light enters the detector 118 after passing through the tissue of the patient 117. In another embodiment, light emitted from the emitter 116 may be reflected by elements in the patent's tissue to enter the detector 118. The detector 118 may convert the received light at a given intensity, which may be directly related to the absorbance and/or reflectance of light in the tissue of the patient 117, into an electrical signal. That is, when more light at a certain wavelength is absorbed, less light of that wavelength is typically received from the tissue by the detector 118, and when more light at a certain wavelength is reflected, more light of that wavelength is typically received from the tissue by the detector 118. After converting the received light to an electrical signal, the detector 118 may send the signal to the monitor 102, where physiological characteristics may be calculated based at least in part on the absorption and/or reflection of light by the tissue of the patient 117.

Additionally the ear sensor 114 may include an encoder 120, which may contain information about sensors (e.g., the emitter 116 and the detector 118) in the ear sensor 114, such as what type of sensor it is (e.g., whether the sensor is a reflectance sensor, a transmittance sensor, etc., and/or whether the sensor is emitting and detecting light at the ear lobe, car canal, etc.) and the wavelengths of light emitted by the emitter 116. This information may allow the monitor 102 to select appropriate algorithms and/or calibration coefficients for calculating the patient's 117 physiological characteristics. The encoder 120 may, for instance, be a memory on which one or more of the following information may be stored for communication to the monitor 102: the type of the sensor 114; the wavelengths of light emitted by the emitter 116; and the proper calibration coefficients and/or algorithms to be used for calculating the patient's 117 physiological characteristics. In one embodiment, the data or signal from the encoder 120 may be decoded by a detector/decoder 121 in the monitor 102.

Signals from the detector 118 and the encoder 120 may be transmitted to the monitor 102. The monitor 102 may include one or more processors 122 coupled to an internal bus 124. Also connected to the bus may be a RAM memory 126 and a display 104. A time processing unit (TPU) 128 may provide timing control signals to light drive circuitry 130, which controls when the emitter 116 is activated, and if multiple light sources are used, the multiplexed timing for the different light sources. TPU 128 may also control the gating-in of signals from detector 118 through a switching circuit 134. These signals are sampled at the proper time, depending at least in part upon which of multiple light sources is activated, if multiple light sources are used. The received signal from the detector 118 may be passed through an amplifier 136, a low pass filter 138, and an analog-to-digital converter 140 for amplifying, filtering, and digitizing the electrical signals the from the ear sensor 114. The digital data may then be stored in a queued serial module (QSM) 142, for later downloading to RAM 126 as QSM 142 fills up. In an embodiment, there may be multiple parallel paths for separate amplifiers, filters, and A/D converters for multiple light wavelengths or spectra received.

In an embodiment, based at least in part upon the received signals corresponding to the light received by detector 118, processor 122 may calculate the oxygen saturation using various algorithms. These algorithms may use coefficients, which may be empirically determined. For example, algorithms relating to the distance between an emitter 116 and various detector elements in a detector 118 may be stored in a ROM 144 and accessed and operated according to processor 122 instructions.

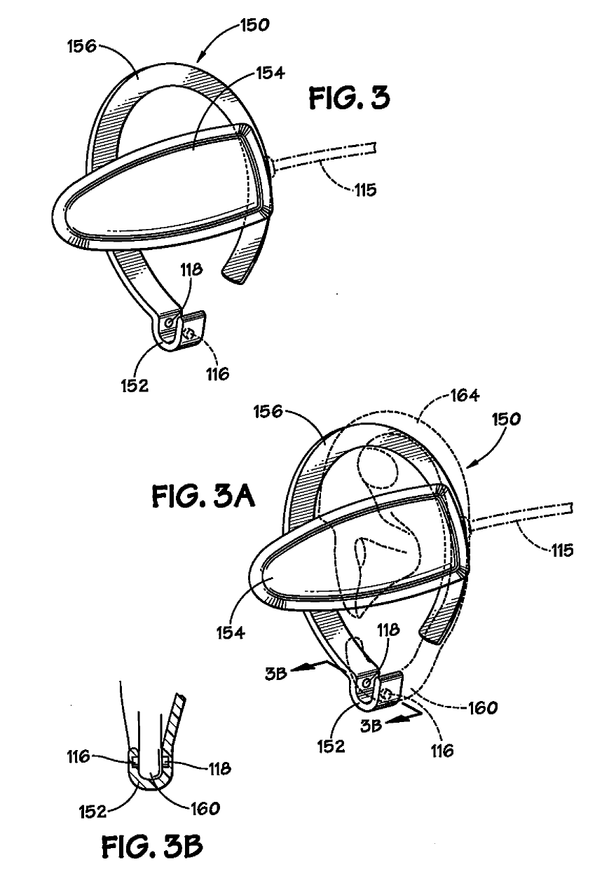

In accordance with the present techniques, embodiments of the ear sensor 114 may have different components configured to optimize the surface contact between the tissue and the sensors of the ear sensor 114. In one or more embodiments, the ear sensor 114 may include a component for retaining the ear sensor 114 to the ear of the patient 117 (referred to as the “retaining component”). An ear sensor 114 may also have a component including an emitter 116 and detector 118 (referred to as the “sensing component”) that is predominantly configured for obtaining a physiological signal. The car sensor 114 may be designed such that the surface contact between the sensing component and the tissue of the patient 117 is predominantly configured to provide an optimized signal. More specifically, as the retaining component may be configured to retain the ear sensor 114 to the ear, the sensing component does not have to apply a retaining force for retaining the ear sensor 114 to the patient 117. Examples of different embodiments of sensing ear sensors 114 in accordance with the present techniques are depicted in FIGS. 3-8.

FIG. 3 depicts an ear sensor 150 having a sensing component 152, a processing component 154, and a retaining component 156. The sensing component 152 may include one or more emitters 116 which direct light through tissue, and the light passing through the tissue is received at one or more detectors 118. The received light, such as a pulse oximetry signal, may be processed at the processing component 154. As depicted in FIG. 3A, the ear sensor 150 may be held to a person's ear 164 (represented in dotted lines) to obtain a pulse oximetry signal from the person's earlobe 160. As illustrated in FIG. 3B, the emitter 116 may direct light through the earlobe 160, and the detector 118 may receive the light which passes through the earlobe 160.

In one embodiment, the sensing component 152 may be an extension from the processing component 154 configured to clip to the ear lobe 160. The sensing component 152 may have opposing members, and one member may have one or more emitters 116 while the opposing member may have one or more detectors 118. The opposing members may be configured to be substantially opposing (i.e., across from each other), such that the emitter(s) 116 on one member may direct a light and the detector(s) 118 on the other member may receive the light passing through the ear lobe 160. The opposing members of the sensing component 154 may be in the form of a clip, clamp, hinge, or spring, or any configuration that would allow the opposing members to apply an appropriate amount of force against the ear lobe 160. For example, the force should be sufficient to effectively couple the emitter 116 and detector 118 to the tissue, but not so much as to exsanguinate the tissue.

The processing component 154 may include various signal processing components discussed with respect to the monitor 102 of the pulse oximeter 100 in FIG. 2. The level of processing in the processing component 154 may vary in embodiments of the present techniques, and may depend on desired characteristics of the ear sensor 150 (e.g., size, wireless capabilities, system configurations, etc.). In other words, some of all of the processing capabilities of the monitor 102 may be carried out by the processing component 154. For example, the processing component 154 may digitize a signal and transmit the digitized signal to the monitor 102, or the processing component 154 may calculate data corresponding to physiological parameters (such as SpO₂, pulse rate, etc.), and may transmit the data to be displayed on the monitor 102. In some embodiments, the processing component 154 may calculate physiological data and provide the data to the user aurally.

The retaining component 156 may retain the ear sensor 150 to the ear 164, and may include a member designed to be malleable and flexible such that the member may maintain contact to the ear 164. For example, the retaining component 156 may be a curved member extending from the ear sensor 150, and may be configured to apply a retaining force to the ear 164, which may be a force sufficient to retain the ear sensor 150 to the ear 164. One purpose of the retaining component 156 may be to retain the ear sensor 150 to the ear 164 so that the sensing component 152 need not be configured to apply a retaining force, which may be greater than a force desired for obtaining an improved signal.

The cable 115 may enable communication between the ear sensor 150 and an external device (e.g., a monitor 102, as in FIG. 1). For example, the pulse oximetry signal, or any data obtained after processing the pulse oximetry signal, may be transmitted to an external device for further processing and/or display. While a cable 115 has been illustrated in FIG. 3, any ear sensor of the present techniques (including the ear sensor 150 of FIG. 3) may transmit information wirelessly, as depicted in FIG. 1A, and may not require a cable 115. For example, the processing component 154 or the retaining component 156 may include an antenna for wireless transmission.

Furthermore, an ear sensor 150 may also be a self-sufficient device with internal processing, and may function without communication with an external device. For example, the processing component 154 in the ear sensor 150 may calculate physiological data, and may also output the physiological data directly. In some embodiments, the ear sensor 150 may provide data to a user aurally, or visually (e.g., the ear sensor 150 may have a display component capable of displaying SpO₂, pulse rate, temperature, or blood pressure, for example).

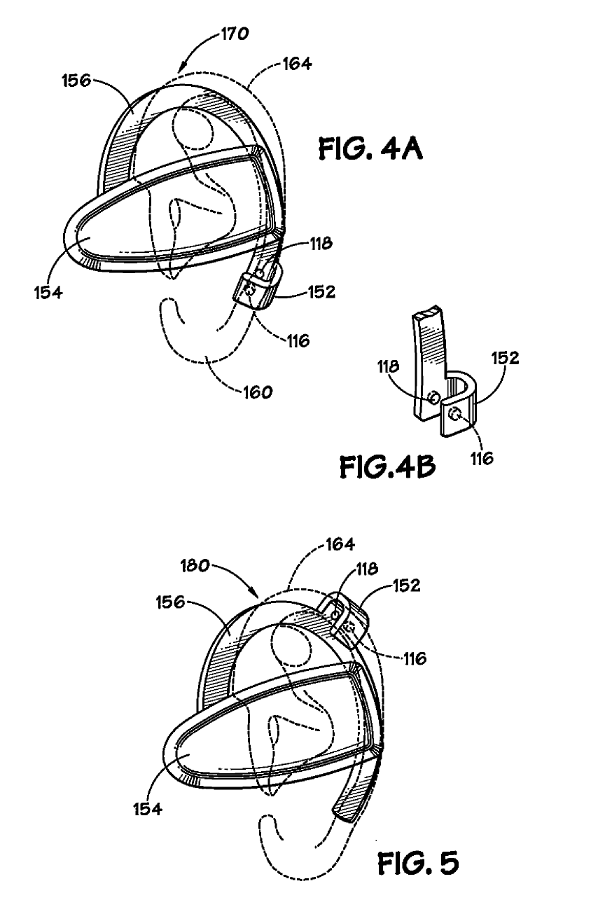

As depicted in FIGS. 4A and 4B, another embodiment of the present techniques may include a sensing component 152 extending from the retaining component 156 of the ear sensor 170. The ear sensor 170 is illustrated in FIG. 4A as fitting over a person's ear 164. The sensing component 152 of the ear sensor 170 may be configured to obtain a pulse oximetry signal from the ear lobe, or any part of the ear 164. A more detailed depiction of a side view of the sensing component 152 is illustrated in FIG. 4B, where one or more emitters 116 may be positioned across from one or more detectors 118 to receive light transmitted through the ear 164. As the ear sensor 170 has a retaining component 156 configured to maintain the ear sensor 170 on the ear 164, no additional force may be utilized from the sensing component 152 to maintain the device 170 on the car. Rather, the sensing component 152 may be configured to apply the amount of force desired to receive an optimal pulse oximetry signal. For example, as discussed with respect to the sensor 150 of FIG. 3, the sensing component 152 of the device 170 may be a clip with opposing members each having either one or more emitters 116 or one or more detectors 118. The opposing members may be held against a part of the ear (e.g., the ear lobe 160) with a clip (or clamp, or other configuration) designed to force the opposing members against the ear lobe 160 such that an optimized pulse oximetry signal may be obtained.

As depicted in FIG. 5, the sensing component 152 may extend from any part of the retaining component 156, and it may be configured to direct and receive light from emitter(s) 116 and detector(s) 118 through any part of the ear 164 from which a pulse oximetry signal, or any other signal which may result in physiological data, may be taken.

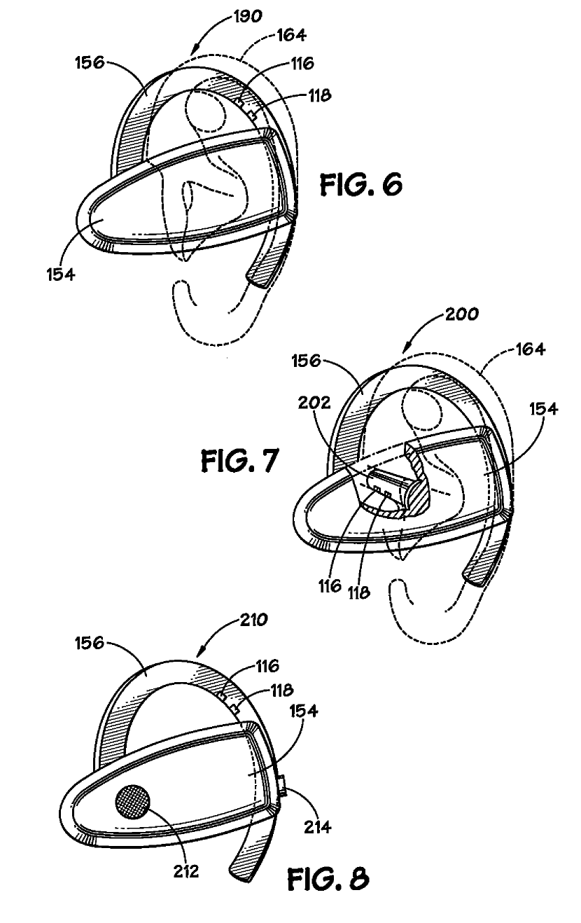

Furthermore, in accordance with one or more embodiments of the present techniques, different types of emitters and detectors may be implemented to receive a pulse oximetry signal from different parts of the ear 164, as depicted in the ear sensor 190 illustrated in FIG. 6. For example, a reflectance style emitter(s) 116 and detector(s) 118 may be configured in the ear sensor 190 (e.g., in the retaining component 156, as illustrated). The emitter(s) 116 may direct light to the tissue of the ear (represented in dotted lines), and the detector(s) 118 may receive the light reflected from the emitter(s) 116 by the ear 164. This reflected light received at the detector(s) 118 may be the pulse oximetry signal, which may be further processed, stored, or output by the processing component 154. As discussed, the pulse oximetry signal may be used to determine certain physiological data, including oxygen saturation in hemoglobin, pulse rate, etc. Further, any ear sensor in accordance with the present techniques, including the ear sensor 190, may include other sensing components to obtain other physiological data, such as temperature, blood pressure, etc.

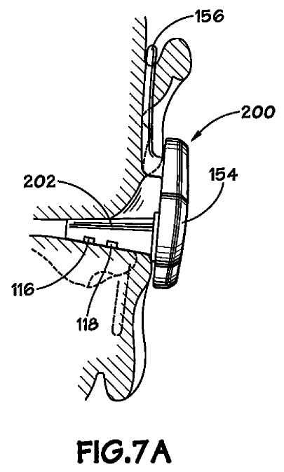

In another embodiment, a reflectance style emitter(s) 116 and detector(s) 118 may be configured on a sensing component 202 configured to fit into a portion of the canal of a person's ear 164, as depicted in the ear sensor 200 of FIG. 7. The emitter(s) 116 may direct light towards a wall of the ear canal, and the detector(s) 118 may receive the light reflected from the tissue in the car canal. Emitting and detecting the light to and from the ear canal tissue may be improved when the emitter(s) 116 and detector(s) 118 have a certain surface contact (i.e., an amount of force between the sensing component 202 and the ear canal tissue). Thus, the sensing component 202 may be in the form of an insert, a plug, a probe, a cushion, or any other component configured to provide a surface contact between the ear canal and the emitter(s) 116 and detector(s) 196. The ear sensor 200 may have a retaining component 156 configured to retain the car sensor 200 to the ear 164. Thus, as the retaining component 156 is configured to support the weight of the ear sensor 200 to the ear 164, no other component of the ear sensor 200 may be used to support the weight of the car sensor 200. As discussed, the sensing component 202 may be configured to have a surface contact between the ear canal and the emitter(s) 116 and detector(s) 118 that produces an optimized pulse oximetry signal.

In one or more embodiments, including all embodiments previously discussed, an car sensor may have varying levels of processing functions and capabilities. For example, in some embodiments, as depicted in FIG. 8, an ear sensor 210 may be capable of alerting a person audibly, via a speaker 212. The ear sensor 210 may be programmed to alert the wearer (or any other person monitoring the wearer of the ear sensor 210) of a status associated with the wearer's pulse oximetry signals. For instance, the ear sensor 210 may produce an audible alert (a beep, a ring, an automated sound, a customizable sound, etc.) based on a status of a physiological parameter. In some embodiments, the ear sensor 210 may produce a beep when a wearer's oxygen saturation level drops beneath a threshold level. Further, the ear sensor 210 may include a button 214, and a person may engage the button to produce audible information. For instance, when the button is pressed, the ear sensor 210 may inform the wearer (or any person monitoring the wearer) of the wearer's current oxygen saturation level, current pulse rate, or any other physiological data obtained by the ear sensor 210. The physiological data may be delivered via a spoken message, for example, through the speaker 212. In some embodiments, the ear sensor 210 may include a display (not shown), which may display physiological data to the user. For example, a user may remove the ear sensor 210 from the user's ear 164 and display the data by engaging the button 214. The user may engage the button 214 again (or engage a different button) to clear the display and/or resume sensing, and place the ear sensor 210 back on the user's ear 164.

Further, the data may be transmitted to an external device, such as a cell phone, a personal digital assistant (PDA), or any other electronic device which may provide data to the user either aurally or visually. The present techniques may also include incorporating sensors into a hands free headset capable of communicating with a cell phone or a PDA. For example, sensors may be incorporated into a Bluetooth® headset to detect physiological signals. The headset may include processing components to process the detected signals and transmit the data to an external device (e.g., a cell phone).

Though the present disclosure generally discusses pulse oximetry signals, embodiments of the present techniques, an ear sensor configured to obtain an optimized signal, may also provide an improved data related to other physiological parameters, such as temperature, blood pressure, etc., or also data related to ambient parameters around the user. For example, the ear sensor may include temperature sensors, blood pressure sensors, or other biosensors. In some embodiments, the ear sensor may also include sensors for sensing an ambient parameters, including ambient temperature, air pressure, and air composition, etc. The ear sensor may include any sensors which enable monitoring of the wearer's condition. In accordance with the present techniques, the sensors of the ear sensor may have a desired surface contact with the wearer, such that the sensed signals are optimized.

Embodiments of the present techniques may also include processing, displaying, and/or organizing the physiological data on an external device (e.g., the monitor 102 from FIG. 1). The external device may be portable, such as a cell phone, a pager, or a personal digital assistant (PDA), or the external device may be non-portable, such as a computer, or any other device capable of processing, organizing, and/or displaying physiological data based on a physiological signal (e.g., a pulse oximetry signal) received from a sensing ear sensor of the present techniques. The external device may include an application or a user interface which may allow a user of the external device to monitor and analyze physiological parameters of the wearer of the ear sensor 114. In some embodiments, the user of the external device may also be the wearer of the ear sensor 114, or a medical professional, or any other person who monitors the physiological data of the wearer. Further, the ear sensor 114 may communicate with the external device through a wire (e.g., via cable 115), or wirelessly (e.g., FIG. 1A). For example, a person, such as a patient, may be monitored via telemetry by medical staff at a hospital. The patient may be contacted by the hospital if the monitored physiological conditions meet certain criteria.

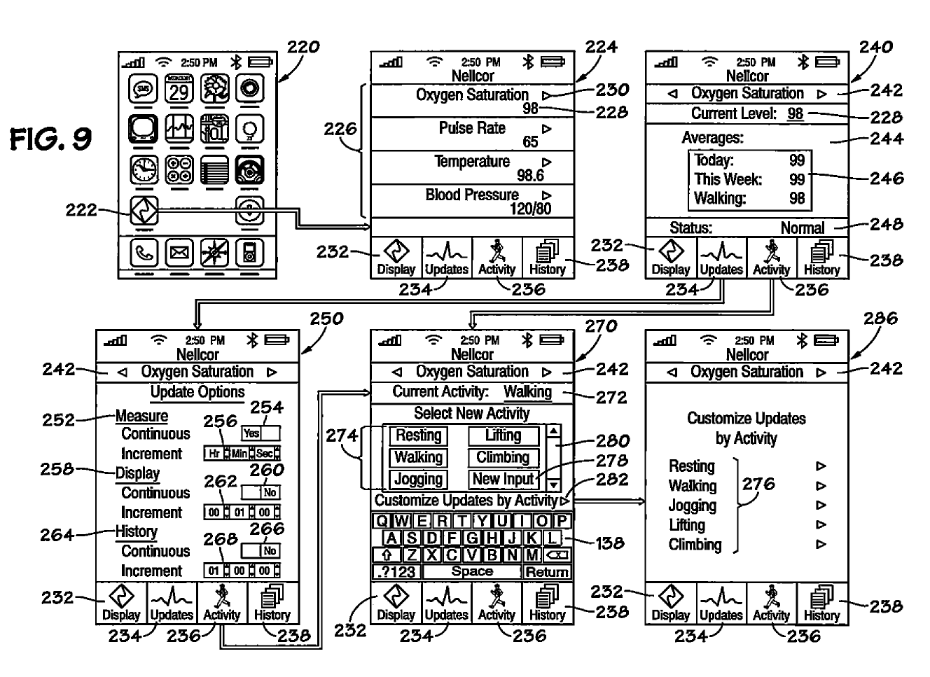

One example of an external device may be the iphone®, available from Apple Inc. of Cupertino, Calif. An external device, such as the iPhone, may have applications configured to enable a user to access sensor data. Further, the application may enable the user to view or analyze the sensor data, or be alerted to some status of a physiological parameter. Depictions of how sensor data may be accessed by a user, using a user interface similar to an iPhone application as an example, are presented in FIGS. 9-11.

Referring first to FIG. 9, a plurality of screen images on an external device depicting a technique for displaying and organizing physiological data based on a physiological signal received at the ear sensor 114 is illustrated. Beginning with the home screen 220, a user may initiate an application for viewing and organizing physiological data obtained through an ear sensor 114 by selecting the graphical icon 222. Upon selecting the graphical icon 222, the screen 224 may be displayed on the external device. The screen 224 may display a listing 276 of various physiological parameters and which may be viewed or organized on the device. For example, the physiological data application 222 may store physiological data which may be obtained from a pulse oximetry sensor, such as oxygen saturation and pulse rate. Further, an ear sensor 114 of the present techniques may include other physiological sensors, including temperature or blood pressure sensors, and may transmit temperature or blood pressure data to be displayed and organized by the physiological data application 222 on an external device. As shown on the screen 224, each physiological parameter in the listing 226 may display a current level. For example, the oxygen saturation 230 of a wearer of the ear sensor 114 may have a current level 228 of 98. In some embodiments, the screen 224 may also display other information of each physiological parameter 226, including wave forms (e.g., a pulse oximetry curve, or an electrocardiogram tracing) or alerts. The screen 224 may further display the graphical buttons 232, 234, 236, and 238. Each of these graphical buttons may correspond to specific functions that may be selected by the user, as will be discussed in further detail.

Each of the parameters may be selected view or analyze the parameter data in further detail. For example, if a user selects oxygen saturation, the user may be navigated to the screen 240 for a more detailed summary of oxygen saturation information. In one embodiment, the user may also use the arrows 242 to scroll through other parameters 226 to view a detailed summary screen 240 for each parameter. The detailed summary screen 240 may display the current level 228 of oxygen saturation, and may also display various averages 224. As will be further explained, the user may select the information to be displayed on the detailed summary screen 240 for each parameter. For example, a user may select to display the current daily or weekly averages. The user may also display an activity average 246, based on whether the user has updated a current activity. The user's current activity may be walking, and the average oxygen saturation level during the current activity 246 may be 98. As will be explained, the user may use the activity graphical icon 236 to update a current activity. The user may also display a status description 248 on the detailed summary screen 240, which may be used to indicate alerts. For example, the status description 248 may indicate that the current oxygen saturation level is normal, or may alert the user that the current level is abnormal or dangerously low.

As discussed, the physiological data application 222 may enable a user to analyze the sensed physiological data, including enabling a user to decide how frequently the physiological data is measured or updated. Measuring physiological data may refer to some combination of sensing a physiological signal (e.g., a pulse oximetry signal) by an ear sensor 114, decoding and/or calculating physiological data (e.g., oxygen saturation level or pulse rate, etc.) based on the signal, and receiving the physiological data (e.g., wireless transmission of data from the car sensor 114) on an external device. In some embodiments, the user's input in the physiological data application 222 may be communicated to the ear sensor 114, and may determine how frequently the sensors measure a physiological parameter. For example, a user may have a measuring option 252 which enables the user to select whether the measuring the oxygen saturation should be continuous. By selecting “yes” on the continuous measuring switch 254, the user may direct the ear sensor 114 to continuously sense a pulse oximetry signal, or may enable the application 222 to continuously receive oxygen saturation data from the car sensor 114. If the user selects “no” on the continuous measuring switch 254, the user may use the increment input 256 to select a time increment (e.g., in hours, minutes, and seconds) for measuring oxygen saturation.

In one embodiment, a user may further use a display option 258 to decide how often the display of the current level 228 of oxygen saturation 230 may be updated. For example, the user may select “yes” on the continuous display switch 260 such that the current level 228 of the oxygen saturation 230 measured will be continuously displayed. The user may also select “no” on the continuous display switch 260, and may instead select a time increment by which the oxygen saturation level display 228 is updated. The increment input 262 may have boxes and scroll bars to allow the user to either input or change a time increment by hours, minutes, and/or seconds.

Some embodiments of the present techniques may also enable a user to record physiological data into “history,” which may refer to a log, database, or some memory component coupled to the external device. Further, the user may select the frequency at which physiological data is recorded into history. For example, the user may use the history update option 264. A continuous history switch 266 may allow the user to select whether oxygen saturation data should be continuously recorded into history, and an increment input 268 may enable a user to select a time increment at which the current level 228 of oxygen saturation 230 is recorded into history. For example, in some embodiments, directing the application 222 to update in time increments, rather than continuously, may save memory space in the history, or processing power. Furthermore, in one or more embodiments, the history may be downloaded onto an external database (such as memory in the user's personal computer, etc.).

Though the update options screen 250 shows update options for oxygen saturation data, in accordance with the present techniques, other data corresponding to other physiological parameters may also have customizable update options. For example, in one embodiment, a user may use the arrows 242 to change physiological parameters, and may change from oxygen saturation to any other parameter 226 to update measurement, display, and history options of the parameter 226.

As illustrated in the update options screen 250, the user may access the graphical icons 232, 234, 236, or 238. For example, from the update options screen 250, the user may use the activity icon 236 to select or input a current activity. The activity screen 270 may enable the user to view the current activity 272, and to select a new activity from an existing list 274 of activities. The list 274 may include buttons labeled with default activities or activities input by a user by a new input button 278. By pressing the new input button 278, the screen 270 may display a text field and a text keyboard interface for typing an activity into the list 274. For example, the user may input certain activities, such as climbing 276 or high altitude training (not illustrated), for which oxygen saturation monitoring may be particularly relevant. In one or more embodiments, the application 222 may be customizable for various activities and users. In addition to using an ear sensor 114 with the application 222 in hospitals or patient monitoring, the present techniques may also apply to various types of physical training. For example, the present techniques may be used for persons living or travelling in high altitude environments, where oxygen saturation levels or other physiological parameters may be important to monitor. Additionally, the present techniques may apply to military training, or any other situation where the tracking of physiological parameters may be relevant. As discussed, an ear sensor 114 of the present techniques may obtain an optimized physiological signal, which may translate into more accurate physiological data, as the sensing component of the ear sensor 114 may be configured to receive an improved signal.

A user may have any number of activities, and may navigate the activity list 274 by using a scroll bar 280. Furthermore, the screen 270 may enable a user to customize update options by activity, via the activity update option 282. By selecting the activity update option 282, the screen 286 may appear, which may enable a user to select from the list 276 of activities. By selecting an activity, the user may be brought to a screen similar to screen 250, where the updating options for each activity 276 may be customized. Thus, the user may be able to determine how frequently physiological data is measured, displayed, or recorded, when the user is engaging in a particular activity 271. The activity update option 282 may affect the activity averages 246 or activity history, as will be further discussed.

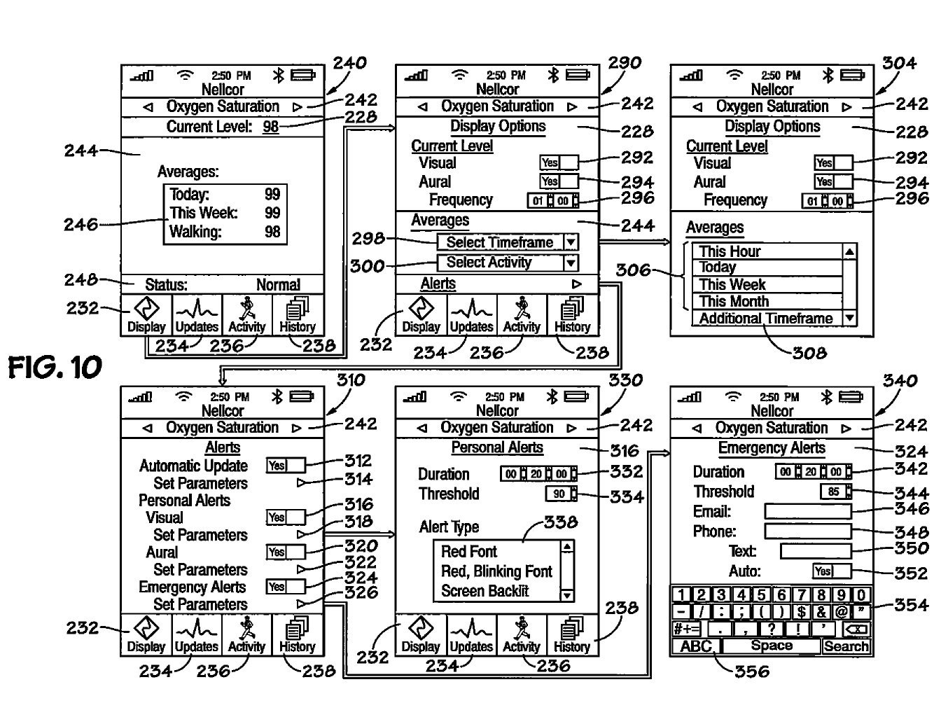

In one or more embodiments, as illustrated in the plurality of screen images of FIG. 10, a physiological data application 222 (as in FIG. 9) may display and organize physiological data, and may further provide various alerts, should a physiological parameter drop or rise to a certain levels. Referring again to the detailed summary screen 240 of oxygen saturation data, a user may select the display graphical icon 232 to access a display options screen 290. As discussed before, because the user has accessed the display graphical icon 232 on the oxygen saturation detailed summary screen 240, the display options screen 290 may show display options for oxygen saturation. The user may also use the arrows 242 to alternate between display options for other physiological parameters 226 (FIG. 9) in the physiological data application 222.

Using oxygen saturation as an example, the user may modify the display options for the current level 228 of oxygen saturation. For example, the current level visual switch 292 may enable the user to select “yes” to display the current level of oxygen saturation. The current level aural switch 294 may enable the external device to produce an audible signal to the user to indicate the current level of oxygen saturation. The user may also use the frequency increment input 296 to select the frequency at which the audible signal is emitted. For example, a user may input 1 hour and 0 minutes into the increment input 296, such that the application 222 causes the device to output an sound (e.g., a voice recording, stating “current oxygen saturation level is 98”) every hour. The aural updates may continue until the user switches the aural switch 294 to “no”.

In one or more embodiments, the user may also modify the displayed averages 244 on the display summary screen 240. For example, the user may access a pull down menu 298 to select a timeframe for average displays 244. The user may also access a pulldown menu 300 to select an activity for average displays 244. As seen in screen 304, accessing the pulldown menu 298 for selecting a timeframe may give the user a list of options 306, such as an average for the current hour, the current day, the current week, or the current month. Additionally, the user may select the additional timeframe option 308 to select an average through a different timeframe. For example, accessing the additional timeframe option 308 may allow the user to also display an average from another month(s).

Referring back to the display options screen 290, the user may also access the alerts option 302, which may allow the user to customize alert settings by selecting different parameters for various types of alerts. For example, in accessing the alert option 302, the user may be directed to the alert screen 310 where the user may have options to customize parameters for various types of alerts. One type of alert may include an automatic update, where the history may be automatically updated with an oxygen saturation level that surpasses certain parameters. An automatic update switch 312 may be switched on, such that an oxygen saturation level that meets certain parameters may be recorded in history, even if the an oxygen saturation level at the time would not otherwise be recorded. For example, if a user has set the application 222 to record his oxygen saturation level 228 every hour, then an updated oxygen saturation level which is measured and/or displayed in between hours may not be recorded in history. However, a user may wish to review the data (e.g., the oxygen saturation level, time of the measure level, etc.) corresponding to abnormal oxygen saturation levels. Configuring the application 222 to automatically update the history whenever an oxygen saturation (or any other physiological parameter) meets certain parameters would enable a user to better review and monitor his physiological data. As will be explained with respect to personal alerts, the user may select the option 314 to set parameters for automatic updates.

Another type of alert may include personal alerts, which may be visual 316 or aural 320. For example, the user may switch the visual alert switch 316 to a “yes,” and may select the set parameters option 318. The user may then be directed to the screen 330 for setting visual alert parameters. In one embodiment, the parameters may include a duration input 332, which may include hour, minute, and second boxes. The parameters may also include a threshold 334, such that falling below a threshold 334 for some duration 332 may result in a type of alert 336. The user may select different types of visual alerts from a pulldown menu 338. For example, if a user's oxygen saturation level falls beneath a certain threshold (e.g., below a safe or healthy range) for a certain duration (e.g., a relevant or significant period of time), the current level display 228 on the oxygen saturation summary screen 240, or the current level display 228 on the data summary screen 224 may be altered to draw the user's attention (e.g., red, blinking font for the current level 228). Alternatively, the user may select to alter other visual settings of the external device, even if the user is not in the physiological data application 222 (e.g., backlight the screen of the external device).

Referring again to the main alerts screen 310, a user may also switch the aural personal alert switch 320 to “yes,” and similar to the parameter settings 318 for visual alerts 316, the user may select the set parameters option 322 to set parameters such as duration or threshold, for example, which would lead to an aural alert 320. The aural alert parameters 322 may be the same or different from the visual alert parameters 318. For example, the user may input different parameters, such as a lower threshold or a longer duration, such that the aural alert 320 indicates a more serious or urgent low in the user's oxygen saturation level. The aural alert 320 may have alert types such as a ring, a beep, or a voice recording indicating the current level 228 of the user's oxygen saturation. The aural alert may also be in the form of a phone call, such that the alert may be less conspicuous to others around the user, and may still draw the user's attention to the user's oxygen saturation level.

In some embodiments, another type of alert may include emergency alerts 324, which may allow the user to automatically contact another person or another device when the user's oxygen saturation meets certain parameters 326. The user may set the emergency alert switch 324 to “yes,” and may set emergency alert parameters 326. As depicted in emergency alert screen 340, and similar to the parameter settings 318 in the visual personal alerts screen 330, the user may set the duration 342 and the threshold 344 for activating an emergency alert 324. The threshold 344 may be set based on an oxygen saturation level which may require immediate medical attention. If a user's oxygen saturation level meets the emergency alert parameters 326 (e.g., the oxygen saturation level falls to 85 or below for longer than 20 minutes), an external person or device may be contacted. In one or more embodiments, the ear sensor 114 may include or enable a global positioning system (GPS) such that the user may be located if the user requires medical attention. For example, if the user's physiological condition meets emergency alert parameters, medical personnel may be able to locate the user even if the user is unconscious and/or unable to seek medical attention.

In setting up the physiological data application 222, the user may input information such as an emergency contact email 346 or emergency phone number 348. For example, the email address 346 may have a text box, and a user may input one or more email addresses by using a text keypad 354. The outgoing email may be automated, and may contain information identifying the user and the user's emergency status (not shown). The user may also input a phone number 348, and may use the toggle key 356 to change between a text keypad and a numeric keypad 354. The user may also enter a text message 350 for the phone number 348, or the user may switch an automated voice recording to “yes,” indicating that the voice recording will play once the phone line is answered. For example, the voice recording may indicate the user's identification and emergency status. In other embodiments of the present techniques, other types of alerts may be customized, and other parameters may be used to define an alert. Furthermore, in the alert screens 310, 330, and 340, the user may use the arrows 242 to switch between different physiological parameters, such that the user may efficiently set alert parameters for various physiological conditions.

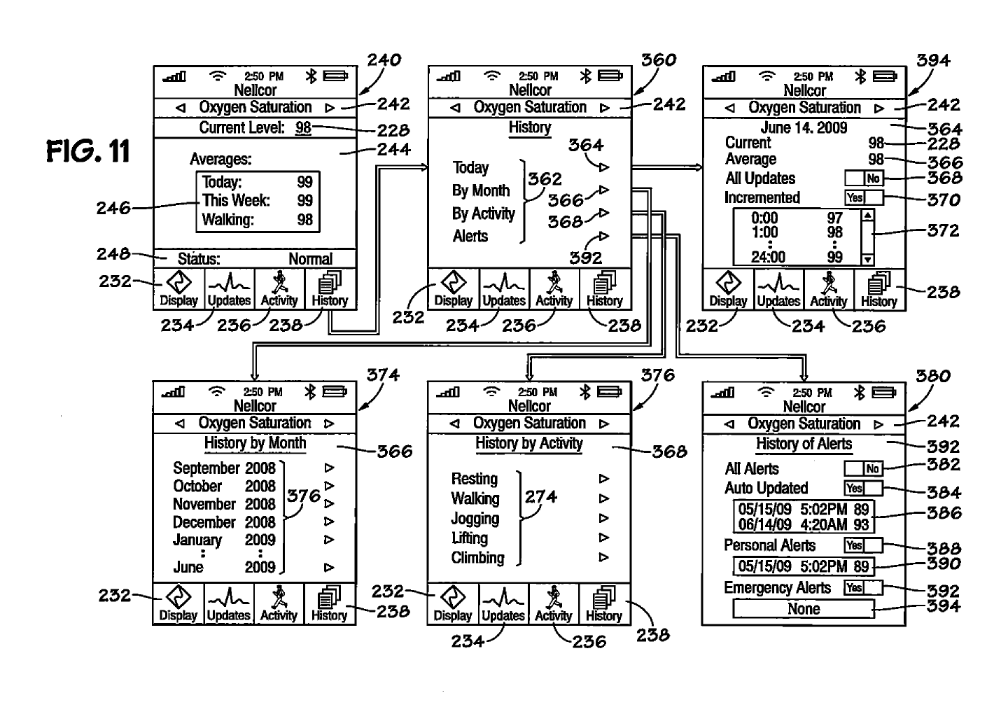

As previously mentioned, the user's physiological data may be recorded into “history,” which may refer to a log, database, or some memory component coupled to the external device capable of holding the user's physiological data. In one or more embodiments, as illustrated in the plurality of screen images of FIG. 11, a physiological data application 222 (as in FIG. 9) may store physiological data, and may further provide organized and/or searchable categories of data history. Referring again to the oxygen saturation detailed summary screen 240, the user may select the history graphical icon 238, and may be directed to the history screen 360 of the user's oxygen saturation levels. As discussed before, the user may also access the history of other physiological data in the list of parameters 226 by using the arrows 242. The history screen 360 may include history categories 362. For example, the user may access the history of the current day 364, and may be brought to the screen 394 where the current day's history 364 may have information such as a current oxygen saturation level 228 and a daily average 366. Further, the screen 394 may allow the user to display all updates, or all recorded oxygen saturation levels of that day by switching the all updates switch 368 to “yes.” The all updates option may include automatically recorded oxygen saturation levels, including automatic updates where levels reached certain alert parameters. The user may also switch the incremented switch 370 to “yes,” which may display a scrollable history 372 of the oxygen saturation levels taken at set increments during the current day 364.

The user may also view history by month 366, and as depicted on the screen 374, the history by month option 366 may display a list 376 of oxygen saturation levels organized by month. A user may select a month from the list 376 to view in further detail. The oxygen saturation history may also be organized by activity 368, such that a user may select detailed history of oxygen saturation levels recorded for any activity on the list 274 of activities in the screen 376. The history may further be organized by alerts 392, and a user may access a history of recorded alerts 392 in the screen 380. The history of alerts screen 380 may have display options, including an all alerts switch 382 which may display all recorded oxygen saturation levels that have been recorded because the level met some alert parameters. The user may select “no” to not display all alerts, and may instead select to display the history of separate alerts. For example, the auto updated history switch 384 may be switched to “yes” to display a history 386 of oxygen saturation levels that have been recorded for meeting certain automatic update parameters 314 (as in FIG. 10). The personal alerts history switch 388 may be switched to “yes” to display a history 390 of oxygen saturation levels recorded for meeting certain personal alert parameters 318 or 322. As depicted, since any of the alerts may have overlapping parameters, an oxygen saturation level recorded as a personal alert may also be recorded as an automatic update (e.g., the oxygen saturation level of 89 on May 15, 2009). The emergency alerts history switch 392 may be switched to “yes” to display a history 394 of oxygen saturation levels that have been recorded for meeting certain emergency alert parameters 326. As depicted, a history 394 of emergency alerts may have no history, as emergency alerts may be set to a lower threshold and may occur less frequently. In some embodiments, a user may select the display graphical icon 232 from any of the history screens 360, 394, 374, 374, and 380 to modify the information displayed on the daily history screen 394. The display options may be similar to the display options screen 290, as discussed in FIG. 10.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. An ear sensor comprising:
a retaining component configured to retain the ear sensor to an ear; and
a sensing component coupled to the retaining component, wherein the sensing component is configured to retain a sensor to the ear and receive a physiological signal.

2. The ear sensor, as set forth in claim 1, wherein the sensing component comprises:
an emitter adapted to transmit light into tissue; and
a light detector configured to receive the transmitted light from the tissue, wherein the physiological signal comprises the transmitted light received by the light detector.

3. The ear sensor, as set forth in claim 2, wherein the sensing component comprises opposing members configured to apply force against opposing sides of a portion of the ear, wherein one member of the opposing members comprises the emitter, and the other member of the opposing members comprises the light detector.

4. The ear sensor, as set forth in claim 1, wherein the sensing component is configured to retain the sensor to the ear with a force that optimizes the physiological signal.

5. The ear sensor, as set forth in claim 4, wherein the force is based substantially on optimizing comfort for a user of the ear sensor.

6. The ear sensor, as set forth in claim 1, comprising a processing component configured to process the physiological signal to produce digitized physiological data.

7. The ear sensor, as set forth in claim 6, comprising an output configured to transmit the digitized physiological data to an external device.

8. The ear sensor, as set forth in claim 1, comprising an output configured to transmit the physiological signal to an external device.

9. The ear sensor, as set forth in claim 8, wherein the external device comprises a processor configured to process the physiological signal to produce digitized physiological data.

10. The ear sensor, as set forth in claim 1, wherein the physiological signal is processed to produce data including one or more of oxygen saturation level, pulse rate, temperature, and blood pressure.

11. The ear sensor, as set forth in claim 10, wherein the data is displayed by an external device configured to enable a user to organize and analyze the displayed data.

12. The ear sensor, as set forth in claim 11, wherein the external device is a cell phone, a personal digital assistant, or any other portable device.

13. A physiological monitoring system comprising:
an ear sensor comprising:
an emitter configured to transmit light into tissue;
a detector configured to receive the light from the tissue; and
a retaining component configured to retain the ear sensor to a wearer's ear; and
an external device coupled to the ear sensor, wherein the external device is configured to display physiological data based on the light received at the detector of the ear sensor.

14. The physiological monitoring system of claim 13, wherein the ear sensor comprises a processor configured to produce physiological data from the light received at the detector.

15. The physiological monitoring system of claim 13, wherein the external device comprises a processor configured to produce physiological data from the light received at the detector.

16. The physiological monitoring system of claim 13, wherein the ear sensor is configured to apply the emitter and the detector to a lobe portion of the wearer's ear.

17. The physiological monitoring system of claim 13, wherein the ear sensor is configured to apply the emitter and the detector to a canal portion of the wearer's ear.

18. The physiological monitoring system of claim 13, wherein the ear sensor is configured to apply the emitter and the detector to any portion of the wearer's ear such that physiological data is obtained from the light received at the detector.

19. The physiological monitoring system of claim 13, wherein the ear sensor is configured to apply the emitter and the detector to the wearer's ear with a force, wherein the applied force is predominantly based on optimizing the light received at the detector.

20. The physiological monitoring system of claim 19, wherein the applied force is less than a retaining force applied by the retaining component to retain the ear sensor to the wearer's ear.

21. The physiological monitoring system of claim 13, wherein the external device is wirelessly coupled to the ear sensor.

22. The physiological monitoring system of claim 13, wherein the external device allows a user to organize the physiological data based on an activity of the wearer.

23. The physiological monitoring system of claim 13, wherein the external device allows a user to set parameters for one or more alerts based on a status of the physiological data.

24. The physiological monitoring system of claim 13, wherein the external device allows a user to determine a frequency of how often the physiological data is recorded.

25. The physiological monitoring system of claim 13, wherein the external device comprises a user interface which allows a user to customize the display of the physiological data based on an activity of the wearer.

26. The physiological monitoring system of claim 13, wherein the external device comprises a user interface which allows a user to monitor the physiological data based on an activity of the wearer.
